(19) United States
(12) Patent Application Publication
(10) Pub. No.: US 2007/0071805 A1
(43) Pub. Date: Mar. 29, 2007

(54) Treatment of inflammation and vascular abnormalities of the eye

(75) Inventors: Arkady Mandel, Mississauga (CA); Patrick L. McGeer, Vancouver (CA); Charles Palmer, Hershey, PA (US); Adele Deering, Dublin (IE); Anthony Ernest Bolton, Dublin (IE)

Correspondence Address: FOLEY & LARDNER LLP, 1530 PAGE MILL ROAD, PALO ALTO, CA, 94304, US

(73) Assignee: Vasogen Ireland Ltd.

(21) Appl. No.: 11/527,299

(22) Filed: Sep. 25, 2006

Related U.S. Application Data
(60) Provisional application No. 60/721,009, filed on Sep. 26, 2005.

(51) Int. Cl. A61K 31/685; A61K 9/127

(52) U.S. Cl. 424/450; 977/907; 514/78

(57) ABSTRACT

Inflammation and vascular abnormalities of the eye, including those related to ischemia, its prophylaxis and its alleviation, are treated by administration to the mammal of small amounts of phosphate-glycerol group presenting bodies such as phosphatidylglycerol liposomes.

### FIELD OF THE INVENTION

This invention relates to inflammation and vascular abnormalities of the eye in a mammalian patient, including those related to ischemia, its prophylaxis and its alleviation.

### BACKGROUND OF THE INVENTION

The eyeball has three layers: the inner retina, which contains the photoreceptors; the middle uvea (choroids, ciliary body, and iris); and the outer sclera, which includes the transparent cornea. The eyeball also contains two cavities, namely a smaller anterior cavity in front of the lens, divided by the iris into an anterior chamber and a posterior chamber, both filled with watery aqueous humor; and a larger posterior cavity, behind the lens, which contains the jelly-like vitreous body (humor). The lens is behind the iris, held in place by the ciliary body and suspensory ligaments. The visible portion of the sclera is covered by the conjunctiva, a membrane which continues as the lining of the eyelids. Extrinsic muscles move the eyeball.

The eye is generally regarded as an immune privileged site. While many of its component parts contain blood vessels and are supplied with blood flow continuously from the body's circulatory system, active components of the body's immune system do not enter the eye, to an extent sufficient to incorporate the eye into the body's normally operating immune system. In order to obtain satisfactory efficacy in the treatment of disorders of the eye, including inflammation, and vascular abnormalities, independent of routes of administration, the drugs or active pharmaceutical agent, or at least the effect of the drugs or active pharmaceutical agent, must be able to cross/penetrate the different blood-tissue barriers, including blood-retina barrier.

Many of the physiological components of the eye are subject to disorders involving inflammation and/or ischemia or vasculature abnormalities. These disorders, grouped under the heading “inflammatory and/or vascular disorders of the eye”, include, but are not limited to, the following:

(a)—“Diabetic retinopathy—retinal damage in patients with diabetes mellitus, the chronic metabolic disorder marked by hyperglycemia. Diabetes mellitus may be of Type 1, in which the body's pancreas fails to produce insulin, or of Type 2, where the body exhibits insulin resistance along with inadequate insulin secretion to sustain normal metabolism. Both types lead to damage of the retina, known as diabetic retinopathy. “Retinopathy” is the general term applied to disorders of the retina.

Diabetic retinopathy is a complication of diabetes that affects the vasculature of the retina. There is increasing evidence that inflammation contributes to the ongoing retinal pathology, and that diabetes causes a local, self-perpetuating inflammatory response within the retina, through activation of endogenous microglia in the retina and recruitment of macrophages, with consequent production of inflammatory mediators. For example, increased levels of inflammatory cytokines IL-1 and IL-6 are found in the retinal tissues of animal models of diabetic retinopathy.

The inflammatory cytokine TNF-α has been implicated in the pathogenesis of diabetic retinopathy. As a consequence, a number of experimental treatments based on blocking their activity have been conducted. Regression of the pathology occurred in 4 cases of diabetic macular edema as a result of anti-TNF-α therapy (Sfikakis et al., Diabetes Care 2005; 28(2): 445-447). Further, TNF-α was shown to be elevated in the serum of diabetic children with retinopathy while serum IL-10 (an anti-inflammatory cytokine) was decreased (Mysliwiec et al., Clin. Biochem. August 2006; 39(8): 851-856).

The deterioration of retinal blood vessels causing loss of blood vessels and leakage into the retina is known as vasculopathy, and leads to visual impairment and may progress to blindness. Diabetes mellitus is the most common cause of blindness among individuals of working age. Retinal blood flow regulation is disrupted very early on in diabetes. The resulting vascular injuries, with their consequent disruption of blood and oxygen supply to the dominant oxygen consuming parts of the retina, have been suggested as significant contributing factors to the progression of diabetic retinopathy (Dao-Yi Yu et. a., “Clinical and Experimental Ophthalmology”, (2001) 29, 164-166).

In addition, macular edema (also known as diabetic macular edema) is a significant clinical symptom of diabetic retinopathy where inflammation is also involved in the pathology. Triamcinolone acetonide, a corticosteroid anti-inflammatory agent, is used to treat diabetic macular edema by intravitreal injection. Diabetic macular edema (DME) occurs after breakdown of the blood-retinal barrier because of leakage of dilated hyperpermeable capillaries and microaneurysms (Ciulla et al., Diabetic Retinopathy and Diabetic Macular Edema: Parthophysiology, Screening, and Novel Therapies, Diabetes Care, (September 2003) Vol. 26, No. 9, 2653-2664). Diabetic patients suffering from diabetic retinopathy can also develop DME. Advanced glycation end products may be involved in perpetuating a pro-inflammatory signalling process, and the interaction between advanced glycation end products and specific receptors for such products may result in disruptions of retinal hemodynamics and/or damage to vascular endothelial cells (Ciulla, supra).

(b)—Uveitis—a non-specific term for intraocular inflammatory disorders. As noted, the uvea is the highly vascularized middle layer of the eyeball, immediately beneath the sclera. It consists of the iris, the ciliary body and the choroids, and forms the pigmented layer. Uveitis usually involves inflammation of some or all of these, but in addition, other, non-uveal parts of the eye such as the retina and the cornea may be involved. It can be associated with infections or diseases of known causes, or unknown causes in which latter case it is known as “endogenous uveitis”, considered to be of autoimmune origin. “Sympathetic uveitis” is severe bilateral uveitis which starts as inflammation of the uveal tract of one eye resulting from a puncture wound. There is evidence that uveitis is inducible by cytokines such as IL-1 (Chiou, J. Ocul. Pharmacol. Ther., 17:189-198, 2001). Some researchers have demonstrated that IL-1 induced uveitis is inhibited by IL-1 blockers (Chiou, J. Ocul. Pharmacol. Ther., 16:407-418, 2000).

(c)—macular degeneration, a loss of pigmentation in the macular region of the retina that produces central visual field loss. It has a significant inflammatory component and some researchers have noted that high blood levels of IL-6 and C-reactive protein (CRP) are associated with a twofold increase in the risk of progression of macular degeneration from early and intermediate stage to advanced stage over a 5 year period.

Age related macular degeneration (AMD) is the commonest type of blindness in the elderly. It is characterized by a loss of retinal pigment epithelial cells and the appearance of drusen at the interface between the choroid and the retina. There is no effective treatment for the dry form of AMD which makes up 85% of cases. There is a prominent inflammatory component to AMD, particularly in the region of drusen. Inflammatory markers such as C-reactive protein, activated microglia and activated complement components have been identified in association with the lesions. Recently it has been reported that rheumatoid arthritics have a ten fold sparing of AMD which has been attributed to their long term use of anti-inflammatory agents (P L McGeer and J Sibley, Neurobiology of Aging 26 (2005) 1199-1203. This finding is consistent with four other reports where polymorphisms in Factor H, a protective factor against self attack by complement, have been found to have a several fold effect on the risk of AMD (G S Hageman et al PNAS 102 (2005) 7227-7232; R J Klein et al Science 308 (2005) 385-389; J L Haines et al Science 308 (2005) 419-421; A O Edwards et al Science 308 (2005) 421-424).

The inflammatory cytokines IL-6 and TNF-α have been implicated in the pathogenesis of AMD. IL-6 was found to be significantly elevated in the serum of AMD cases while there was a mild increase in TNF-α (Seddon et al., Arch. Opthalmol. 2005; 123: 774-782).

These findings indicate that effective anti-inflammatory treatment of AMD should have a very beneficial effect. There is some evidence that age-related macular degeneration is caused by the induction of nitric oxide synthase and overproduction of nitric oxide (a highly reactive compound which may be involved as a mediator in inflammatory processes) in the choriocapillaris arca (Chiou, 2001).

### BRIEF REFERENCE TO THE PRIOR ART

Each of the different manifestations of inflammation of the eye has its currently recommended treatment.

The development and progression of diabetic retinopathy can be reduced by tight (narrow) glycemic control and tight blood pressure control. However, treatments of diabetic retinopathy to date have involved direct treatment of the affected eyes. Laser treatment is used for treatment of diabetic retinopathy. Laser treatment for macular edema reduces the incidence of doubling of visual angle (causing partial sight) in the affected eye by over 50% at two years. Intraocular corticosteroids have been utilized with some success in selected patients, as a treatment of inflammation experienced in the earliest stages of diabetic retinopathy. This involves the use of a long-acting corticosteroid, such as triamcinolone acetonide, injected into the vitreous cavity by way of a very tiny needle, and topical anesthesia. Triamcinolone acetonide has also been used to treat diabetic macular edema by intravitreal injection. There have also been reports of the use of corticosteroids surgically implanted inside the eye to allow constant release of the medication, in a sustained release drug delivery device.

An animal model is available for the study of diabetic retinopathy. This is the streptozotocin-induced diabetic rat model. Diabetes is induced in adult laboratory rats by intraperitoneal or intravascular injection of streptozotocin (STZ). Within 3-5 days of injection, the rats show plasma glucose levels elevated to the range characteristic of diabetes mellitus. They show many of the early changes in retinal structure and function that are associated with human diabetic retinopathy.

Uveitis is normally treated by administration of corticosteroids and other immunosuppressive agents, but these treatments are not universally effective. High-dose corticosteroids are often effective as a treatment for sympathetic uveitis and such compounds are administered intravitreally. Intravitreal injection of triamcinolone acetonide has also been used to treat uveitis.

Laser photocoagulation is effective in preventing progression to damage of the macula, but causes irreversible scarring of treated regions. Administration of pegaptanib (“Macugen”) by intravitreal injection is an experimental treatment for macular degeneration.

Direct injection into the eye is clearly an undesirable procedure which many patients are reluctant to undergo. There have been few reports of successful treatments of diabetic retinopathy, to date, which do not involve direct injections into the eye.

International patent application publication no. WO/03/061667 Vasogen Ireland Limited discloses liposomes and particles exposing phosphatidylglycerol (PG) on their surfaces, and suggests their use in treatment of inflammation and autoimmune diseases in general, by systemic administration and through modification of the body's immune system.

In view of the above, there is a long-felt need for improved treatments for inflammatory and/or vascular disorders of the eye

There is also a need to provide compositions of matter useful in treating inflammatory and/or vascular disorders in the eye, in human patients

### SUMMARY OF THE INVENTION

It has now been found that inflammation of the eye can be alleviated in a mammalian subject, by administration thereto of an appropriate dose of phosphate-glycerol group presenting bodies, of a size resembling the size of apoptotic mammalian cells or apoptotic bodies. The administration may be systemic, not necessarily directly to the eye, e.g. intramuscular or intravenous administration. Surprisingly, administration of the medicament, produces an anti-inflammatory or vascular disorder reducing effect in the eye, normally regarded as an immune-privileged site and generally protected from systemic effects of the body.

Thus according to one aspect of the present invention, there is provided a process of retarding the development and/or progression of an inflammatory and/or vascular disorder in the eye in a mammalian patient, which comprises administering to the patient an effective amount of pharmaceutically acceptable phosphate-glycerol group presenting bodies, of a size resembling that of apoptotic cells or apoptotic bodies.

In one embodiment, the vascular disorder is an ischemic disorder.

According to another aspect of the invention there is provided use in the preparation or manufacture of a medicament for the treatment or prophylaxis of an inflammatory and/or vascular disorder in the eye in a mammalian patient, of pharmaceutically acceptable phosphate-glycerol group presenting bodies, of a size resembling the size of apoptotic cells or apoptotic bodies.

### BRIEF REFERENCE TO THE DRAWINGS

FIG. 1 is a graphical presentation of the measurements of messenger RNA (“mRNA”) levels of the cytokine IL-1 from the retinas of animals, treated and measured as described in the specific example below;

FIG. 2 is a similar presentation of the measurements of mRNA levels of cytokine IL-6 from the same experiment;

FIG. 3 is a similar presentation of the measurements of mRNA levels of cytokine TNF-α from the same experiment; and

FIG. 4 is a similar presentation of the measurements mRNA levels of cytokine MCP-1 from the same experiment.

FIG. 5 is a similar presentation of the measurements of mRNA levels of cytokine TGF-β2 from the same experiment.

FIG. 6 is a similar presentation of the measurements of mRNA levels of cytokine IL-10 from the same experiment.

### THE PREFERRED EMBODIMENTS

Details of the phosphate-glycerol group presenting bodies useful in the present invention, and their preparation can be found in aforementioned International patent application publication no. WO/03/061667 Vasogen Ireland Limited, and its U.S. counterpart U.S. Ser. No. 10/348,601 Bolton and Mandel, filed Jan. 21, 2003, incorporated herein by reference in its entirety. Suitably, the bodies have a size resembling the size of apoptotic cells or apoptotic bodies. These pharmaceutically acceptable bodies include synthetic and semi-synthetic bodies having shapes which are typically but not exclusively spheroidal, cylindrical, ellipsoidal, including oblate and prolate spheroidal, serpentine, reniform etc., and sizes from about 20 nm to about 500 μm in diameter, preferably measured along its longest axis, and comprising phosphate-glycerol groups on the surface thereof.

The pharmaceutically acceptable bodies have phosphate-glycerol groups, or groups capable of conversion in vivo, of predetermined characteristics on the exterior surface. The structure of these groups may be synthetically altered and include all, part of or a modified version of the original phosphatidylglycerol group. For example, the negatively charged oxygen of the phosphate group may be converted to a phosphate ester group (e.g., L-OP(O)(OR′)(OR″), where L is the remainder of the phosphatidylglycerol group, R′ is —CH₂CH(OH)CH₂OH and R″ is alkyl of from 1 to 4 carbon atoms or hydroxyl substituted alkyl of from 2 to 4 carbon atoms, and 1 to 3 hydroxyl groups provided that R″ is more readily hydrolyzed in vivo than the R′ group; to a diphosphate group including diphosphate esters (e.g., L-OP(O)(OR′)OP(O)(OR″)₂ wherein L and R′ are as defined above and each R″ is independently hydrogen, alkyl of from 1 to 4 carbon atoms, or a hydroxyl substituted alkyl of from 2 to 4 carbon atoms and 1 to 3 hydroxyl groups provided that the second phosphate group [—P(O)(OR″)₂] is more readily hydrolyzed in vivo than the R′ group; or to a triphosphate group including triphosphate esters (e.g., L-OP(O)(OR′)OP(O)(OR″)OP(O)(OR″)₂ wherein L and R′ are defined as above and each R″ is independently hydrogen, alkyl of from 1 to 4 carbon atoms, or a hydroxyl substituted alkyl of from 2 to 4 carbon atoms and 1 to 3 hydroxyl groups provided that the second and third phosphate groups are more readily hydrolyzed in vivo than the R′ group; and the like. Such synthetically altered phosphate-glycerol groups are capable of expressing phosphate-glycerol in vivo and, accordingly, such altered groups are phosphate-glycerol convertible groups.

Phosphatidylglycerol is a known compound. It can be produced, for example, by treating the naturally occurring dimeric form of phosphatidylglycerol, cardiolipin, with phospholipase D. It can also be prepared by enzymatic synthesis from phosphatidylcholine using phospholipase D—see, for example, U.S. Pat. No. 5,188,951 Tremblay, et al. Chemically, it has a phosphate-glycerol group and a pair of similar but different C₁₈-C₂₀ fatty acid chains.

As used herein the term “PG” is intended to cover phospholipids carrying a phosphate-glycerol group with a wide range of at least one fatty acid chains provided that the resulting PG entity can participate as a structural component of a liposome. Preferably, such PG compounds can be represented by the Formula I: 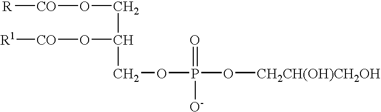
where R and R¹ are independently selected from C₁-C₂₄ hydrocarbon chains, saturated or unsaturated, straight chain or containing a limited amount of branching wherein at least one chain has from 10 to 24 carbon atoms. Essentially, the lipid chains R and R¹ form the structural component of the liposomes, rather than the active component. Accordingly, these can be varied to include two or one such lipid chains, the same or different, provided they fulfill the structural function. Preferably, the lipid chains are from about 10 to about 24 carbon atoms in length, saturated, mono-unsaturated or polyunsaturated, straight-chain or with a limited amount of branching. Laurate (C12), myristate (C14), palmitate (C16), stearate (C18), arachidate (C20), behenate (C22) and lignocerate (C24) are examples of useful saturated lipid chains for the PG for use in the present invention. Palmitoleate (C16) and oleate (C18) are examples of suitable mono-unsaturated lipid chains. Linoleate (C18), linolenate (C18) and arichidonate (C20) are examples of suitable poly-unsaturated lipid chains for use in PG in the liposomes of the present invention. Phospholipids with a single such lipid chain, also useful in the present invention, are known as lysophospholipids. The present invention also extends to cover use of liposomes in which the active component is the dimeric form of PG, namely cardiolipin but other dimers of Formula I are also suitable. Preferably, such dimers are not synthetically cross-linked with a synthetic cross-linking agent, such as maleimide but rather are cross-linked by removal of a glycerol unit as described by Lehniger, Biochemistry, p. 525 (1970) and depicted in the reaction below: 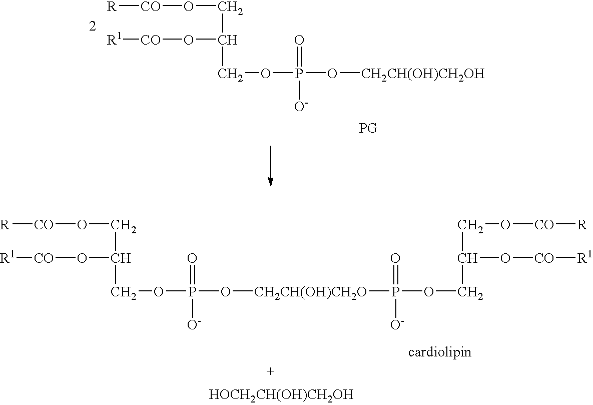
where each R and R¹ are independently as defined above.

The term “phosphate-choline” refers to the group —O—P(═O)(OH)—O—CH₂—CH₂—N⁺(CH₃)₃, which is attached to the remainder of the lipid as shown in the following structure: 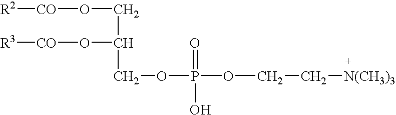
and salts thereof, wherein R² and R³ are independently selected from C₁-C₂₄ hydrocarbon chains, saturated or unsaturated, straight chain or containing a limited amount of branching wherein at least one chain has from 10-24 carbon atoms.

“Phosphate-glycerol group presenting bodies”, as described herein, refers to biocompatible, pharmaceutically-acceptable, three-dimensional bodies having on their surfaces phosphate-glycerol groups or groups that can be converted to phosphate-glycerol groups. By way of example, PG can form the membrane of a liposome, either as the sole constituent of the membrane or as a major or minor component thereof, with other phospholipids and/or membrane forming materials.

Examples of “three-dimensional body portions”, “three-dimensional bodies” or pharmaceutically acceptable bodies” include biocompatible synthetic or semi-synthetic entities such as liposomes, solid beads, hollow beads, filled beads, particles, granules and microspheres of biocompatible materials, natural or synthetic, as commonly used in the pharmaceutical industry or known in the art. The beads may be solid or hollow, or filled with biocompatible material. The term “biocompatible” refers to substances which, in the amount employed, are either non-toxic or have acceptable toxicity profiles such that their use in vivo is acceptable. Likewise the term “pharmaceutically acceptable” as used in relation to “pharmaceutically acceptable bodies” refers to bodies comprised of one or more materials which are pharmaceutically acceptable and suitable for delivery in vivo. Such bodies can include liposomes formed of lipids, one of which is PG. Alternatively, the pharmaceutically acceptable bodies can be solid beads, hollow beads, filled beads, particles, granules and microspheres of biocompatible materials, which comprise one or one or more biocompatible materials such as polyethylene glycol, poly(methylmethacrylate), polyvinylpyrrolidone, polystyrene and a wide range of other natural, semi-synthetic and synthetic materials, with phosphate-glycerol groups attached thereto.

Suitable forms of bodies for use in the compositions of the present invention include, without limitation, particles, granules, microspheres or beads of biocompatible materials, natural or synthetic, such as polyethylene glycol, polyvinylpyrrolidone, polystyrene, and the like; polysaccharides such as hydroxethyl starch, hydroxyethylcellulose, agarose and the like; as are commonly used in the pharmaceutical industry. Preferably, such materials will have side-chains or moieties suitable for derivatization, so that a phosphate-glycerol group, such as PG, may be attached thereto, preferably by covalent bonding. Bodies of the invention may be solid or hollow, or filled with biocompatible material. They are modified as required so that they carry phosphate-glycerol molecules, such as PG on their surfaces. Methods for attaching phosphate-glycerol in general, and PG in particular, to a variety of substrates are known in the art.

Preferred compositions of matter are liposomes, which may be composed of a variety of lipids. Preferably, however, none of the lipids are positively charged. In the case of liposomes, phosphatidylglycerol PG may constitute the major portion or the entire portion of the liposome layer(s) or wall(s), oriented so that the phosphate-glycerol group portion thereof is presented exteriorly, to act as the binding group, and the lipid chain or chains form the structural wall.

Liposomes, or lipid vesicles, are sealed sacs, in the micron or sub-micron range, the walls (monolayer or multilayer) of which comprise suitable amphiphiles forming a monolayer or bilayer surrounding a central chamber, which may be fluid filled. They normally contain an aqueous medium, although for the present invention the interior contents are unimportant, and generally inactive. Phospholipids are amphiphilic molecules (i.e. amphiphiles), meaning that the compound comprises molecules having a polar water-soluble group attached to a water-insoluble hydrocarbon chain. The amphiphiles serving as the layers of the matrix have defined polar and apolar regions. The amphiphiles can include, in addition to PG in this invention, other, naturally occurring lipids used alone with the phospholipid carrying the active group, or in a mixture with another. The amphiphiles serving as the layer(s) of the liposomes can be inert, structure-conferring synthetic compounds such as polyoxyethylene alkylethers, polyoxyethylene alkylesters and saccharosediesters.

Preferably, for use in forming liposomes, the amphiphilic molecules will include one or more forms of phospholipids of different headgroups (e.g., phosphatidylglycerol, phosphatidylserine, phosphatidylcholine) and having a variety of fatty acid side chains, as described above, as well as other lipophilic molecules, such as cholesterol, sphingolipids and sterols.

Liposomes of the invention are typically formed from phospholipid bilayers or a plurality of concentric phospholipid bilayers that enclose aqueous phases. In some cases, the walls of the liposomes may be single layered; however, such liposomes (termed “single unilamellar vesicles”) are generally much smaller (diameters less than about 70 nm) than those formed of bilayers, as described below. Liposomes formed in accordance with the present invention are designed to be biocompatible, biodegradable and non-toxic. Liposomes of this type are used in a number of pharmaceutical preparations currently on the market, typically carrying active drug molecules in their aqueous inner core regions. In the present invention, however, the liposomes are not filled with pharmaceutical preparation, i.e. they are essentially free of non-liposomal, pharmaceutically active components. Accordingly, in a preferred embodiment, the liposomes, as well as other pharmaceutically acceptable bodies, are essentially free of non-lipid pharmaceutically active entities (e.g. <1%) and more preferably are free of non-lipid pharmaceutically active entities. The liposomes are active themselves, not acting as drug carrier.

Such liposomes are prepared and treated so that the active groups are presented exteriorly on the liposomal body. The PG in the liposomes of the preferred embodiments of this invention thus serves as both a ligand and a structural component of the liposome itself.

Thus a preferred embodiment of this invention provides use in preparation of a medicament for the treatment or prophylaxis of an inflammatory and/or vascular disorder in the eye in a mammalian patient, of liposomal bodies which expose or can be treated or induced to expose, on their surfaces, one or more phosphate-glycerol groups to act as binding groups. Such lipids should comprise from 10%-100% of the liposome, with the balance being an inactive constituent, e.g. phosphatidylcholine PC, or one which acts through a different mechanism, e.g. phosphatidylserine PS, or mixtures of such. Inactive co-constituents such as PC are preferred.

Preferred phosphate-glycerol group presenting liposomes of the present invention are constituted to the extent of at least 10% by weight is composed of PG, the balance being phosphatidylcholine (PC) or other such biologically acceptable phospholipid(s), preferably at least 50%, more preferably from 60-100% and most preferably from 70-90%, with the single most preferred embodiment being about 75% by weight of PG.

Mixtures of PG liposomes with inactive liposomes and/or with liposomes of phospholipids acting through a different mechanism can also be used, provided that the total amount of PG remains above the minimum of about 10% and preferably above 60% in the total mixture. According to a preferred feature of the invention, phosphate-glycerol group presenting bodies comprise less than 50%, preferably less than 40%, still preferably less than 25% and even still preferably less than 10% phosphatidyl choline.

The present invention contemplates the use, as phosphate-glycerol group presentng bodies, not only of those liposomes having PG as a membrane constituent, but also liposomes having non-PG membrane substituents that carry on their external surface molecules of phosphate-glycerol, either as monomers or oligomers (as distinguished from phosphatidylglycerol), e.g., chemically attached by chemical modification of the liposome surface of the body, such as the surface of the liposome, making the phosphate-glycerol groups available for subsequent interaction. Because of the inclusion of phosphate-glycerol groups on the surface of such molecules, they are included within the definition of phosphate-glycerol group presenting bodies.

As regards to non-liposomal bodies for use in the present invention, these as noted include biocompatible solid or hollow beads of appropriate size. The biocompatible non-liposomal synthetic or semi-synthetic bodies may be selected from polyethylene glycol, poly(methylmethacrylate), polyvinylpyrrolidone, polystyrene and a wide range of other natural, semi-synthetic and synthetic materials, with phosphate-glycerol groups attached to the surfaces thereof.

Biodegradable polymers are disclosed in the art and include, for example, linear-chain polymers such as polylactides, polyglycolides, polycaprolactones, polyanhydrides, polyamides, polyurethanes, polyesteramides, polyorthoesters, polydioxanones, polyacetals, polyketals, polycarbonates, polyorthocarbonates, polyphosphazenes, polyhydroxybutyrates, polyhydroxyvalerates, polyalkylene oxalates, polyalkylene succinates, poly(malic acid), poly(amino acids), polyvinylpyrrolidone, polyethylene glycol, polyhydroxycellulose, chitin, chitosan, and copolymers, terpolymers and combinations thereof. Other biodegradable polymers include, for example, gelatin, collagen, etc.

Suitable substances for derivatization to attach the phospholipid(s), or portions thereof with groups or binding groups, to three-dimensional bodies are commercially available e.g. from Polysciences Inc., 400 Valley Road, Warrington, Pa. 18976, or from Sigma Aldrich Fine Chemicals. Methods for their derivatization are known in the art. Specific preferred examples of such methods are disclosed in International Patent Application PCT/CA02/01398 Vasogen Ireland Limited, which is incorporated herein by reference.

Liposomes may be prepared by a variety of techniques known in the art, such as those detailed in Szoka et al. (Ann. Rev. Biophys. Bioeng. 9:467 (1980)). Depending on the method used for forming the liposomes, as well as any after-formation processing, liposomes may be formed in a variety of sizes and configurations. Methods of preparing liposomes of the appropriate size are known in the art and do not form part of this invention. Reference may be made to various textbooks and literature articles on the subject, for example, the review article by Yechezkel Barenholz and Daan J. A. Chromeline, and literature cited therein, for example New, R. C. (1990), and Nassander, U. K., et al. (1990), and Barenholz, Y and Lichtenberg, D., Liposomes: preparation, characterization, and preservation, Methods Biochem Anal. 1988, 33:337-462.

Multilamellar vesicles (MLV's) can be formed by simple lipid-film hydration techniques according to methods known in the art. In this procedure, a mixture of liposome-forming lipids is dissolved in a suitable organic solvent. The mixture is evaporated in a vessel to form a thin film on the inner surface of the vessel, to which an aqueous medium is then added. The lipid film hydrates to form MLVs, typically with sizes between about 100-1000 nm (0.1 to 10 microns) in diameter.

A related, reverse evaporation phase (REV) technique can also be used to form unilamellar liposomes in the micron diameter size range. The REV technique involves dissolving the selected lipid components, in an organic solvent, such as diethyl ether, in a glass boiling tube and rapidly injecting an aqueous solution, optionally containing a drug solution to be carried in the interior of the liposome, into the tube, through a small gauge passage, such as a 23-gauge hypodermic needle. The tube is then sealed and sonicated in a bath sonicator. The contents of the tube are alternately evaporated under vacuum and vigorously mixed, to form a final liposomal suspension.

The diameters of the phosphate-glycerol group presenting liposomes, as well as the other pharmaceutically acceptable bodies, of the preferred embodiment of this invention range from about 20 nm to 500 μm, more preferably from 20 nm to about 1000 nm, more preferably from about 20 nm to about 500 nm, and most preferably from about 20 nm to about 200 nm. Such preferred diameters will correspond to the diameters of mammalian apoptotic bodies, such as may be apprised from the art.

One effective sizing method for REVs and MLVs involves extruding an aqueous suspension of the liposomes through a series of polycarbonate membranes having a selected uniform pore size in the range of 0.03 to 0.2 micron, typically 0.05, 0.08, 0.1, or 0.2 microns. The pore size of the membrane corresponds roughly to the median size of liposomes produced by extrusion through that membrane, particularly where the preparation is extruded two or more times through the same membrane. This method of liposome sizing is used in preparing homogeneous-size REV and MLV compositions. U.S. Pat. Nos. 4,737,323 and 4,927,637, incorporated herein by reference, describe methods for producing a suspension of liposomes having uniform sizes in the range of 0.1-0.4 μm (100-400 nm) using as a starting material liposomes having diameters in the range of 1 μm. Homogenization methods are also useful for down-sizing liposomes to sizes of 100 nm or less (Martin, F. J. (1990) In: Specialized Drug Delivery Systems—Manufacturing and Production Technology, P. Tyle (ed.) Marcel Dekker, New York, pp. 267-316.). Another way to reduce liposomal size is by application of high pressures to the liposomal preparation, as in a French Press.

Liposomes can be prepared to have substantially homogeneous sizes of single, bi-layer vesicles in a selected size range between about 0.07 and 0.2 microns (70-200 nm) in diameter, according to methods known in the art. In particular, liposomes in this size range are readily able to extravasate through blood vessel epithelial cells into surrounding tissues. A further advantage is that they can be sterilized by simple filtration methods known in the art. Whilst a preferred embodiment of phosphate-glycerol group presenting bodies for use in the present invention is liposomes with PG presented on the external surface thereof, it is understood that the phosphate-glycerol group presenting body is not limited to a liposomal structure, as mentioned above.

The pharmaceutically acceptable bodies may be suspended in a pharmaceutically acceptable carrier, such as physiological sterile saline, sterile water, pyrogen-free water, isotonic sterile saline, and phosphate buffer sterile solutions (e.g. sterile aqueous solutions comprising phosphate buffer), as well as other non-toxic compatible substances used in pharmaceutical formulations, such as, for example, adjuvants, buffers, preservatives, and the like. Preferably, the pharmaceutically acceptable bodies are constituted into a liquid suspension in a sterile biocompatible liquid such as buffered sterile saline and administered to the patient by any appropriate route which exposes it to one or more components of the immune system, such as oral, nasal, rectal, topical, intra-arterial, intravenous or subcutaneous or most preferably intramuscular administration.

It is contemplated that the pharmaceutically acceptable bodies may be freeze-dried or lyophilized so that they may be later re-suspended for administration. This invention therefore also includes a kit of parts comprising lyophilized or freeze-dried phosphate-glycerol group presenting bodies and a pharmaceutically acceptable carrier, such as physiological sterile saline, sterile water, pyrogen-free water, isotonic saline, and phosphate buffer solutions, as well as other non-toxic compatible substances used in pharmaceutical formulations. Such a kit may optionally provide injection or administration means for administering the composition to a subject.

A preferred manner of administering the pharmaceutically acceptable bodies to the patient is a course of injections, administered daily, several times per week, weekly or monthly to the patient, over a period ranging from a week to several months or more. The frequency and duration of the course of the administration is likely to vary from patient to patient, and according to the severity of the condition being treated, and whether the treatment is intended as prophylactic, therapeutic or curative. Its design and optimization is well within the skill of the attending physician. Intramuscular injection is most preferred. One currently preferred dosage schedule is a daily injection for six successive days, followed by a booster injection monthly. It is within routine testing to extrapolate such dosing regimens to other mammalian species. One particular injection schedule is an injection, via the gluteal muscle, of an appropriate amount of bodies on day 1, a further injection on day 2, a further injection on day 14, and then “booster” injections at monthly intervals, if appropriate. Another injection schedule is 6 daily injections and booster injections every 2-4 weeks. The quantities of phosphate-glycerol group presenting bodies to be administered will vary depending on the identity and characteristics of the patient. It is important that the effective amount of phosphate-glycerol group presenting bodies is non-toxic to the patient.

The most effective amounts are unexpectedly small. When using intra-arterial, intravenous, subcutaneous or intramuscular administration of a liquid suspension of phosphate-glycerol group presenting bodies, it is preferred to administer, for each dose, from about 0.1-50 ml of liquid, containing an amount of phosphate-glycerol group presenting bodies generally equivalent to 10%-1000% of the number of leukocytes normally found in an equivalent volume of whole blood or the number of apoptotic bodies that can be generated from them. Generally, the number of phosphate-glycerol group presenting bodies administered per delivery to a human patient is in the range from about 500 to about 3×10¹⁴ (about 30 mg by weight at the highest end of the range), preferably from about 5,000 to about 500,000,000, more preferably from about 10,000 to about 10,000,000, and most preferably from about 200,000 to about 2,000,000.

The correlation between weights of liposomes and numbers of liposomes is derivable from the knowledge, accepted by persons skilled in the art of liposomal formulations, that a 100 nm diameter bilayer vesicle has about 81,230 lipid molecules per vesicle, distributed approximately 50:50 between the layers (see Harrigan—1992 University of British Columbia PhD Thesis “Transmembrane pH gradients in liposomes (microform): drug-vesicle interactions and proton flux”, published by National Library of Canada, Ottawa, Canada (1993); University Microfilms order no. UMI00406756; Canada no. 942042220, ISBN 0315796936). From this one can calculate, for example, that a dose of 5×10⁸ vesicles, of the order of the dose used in the specific in vivo examples below, is equivalent to 4.06×10¹³ lipid molecules. Using Avogadro's number for the number of molecules of lipid in a gram molecule (mole), 6.023×10²³, one determines that this represents 6.74×10⁻¹¹ moles which, at a molecular weight of 747 for PG is approximately 5.04×10⁻⁸ gm, or 50.4 ng of PG for such dosage.

According to one feature of the invention, the number of such bodies administered to an injection site for each administration is believed to be a more meaningful quantification than the number or weight of phosphate-glycerol group presenting bodies per unit of patient body weight. Thus, it is contemplated that effective amounts or numbers of phosphate-glycerol group presenting bodies for small animal use may not directly translate into effective amounts for larger mammals on a weight ratio basis. The person skilled in the art could readily extrapolate from the data and other information contained herein to arrive at appropriate dosing for other mammals.

One preferred utility of the present invention is in treatment of diabetic retinopathy. Experimental evidence in preclinical work using the process and compositions of the present invention indicates a significant decrease in the retinas of animals suffering from diabetic retinopathy of the inflammatory cytokines IL-1 and IL-6 and the inflammatory chemokine MCP-1, inflammatory mediators known to be factors in the development and progression of diabetic retinopathy in mammals.

The term diabetic retinopathy includes all conditions associated or arising therefrom, including but not limited to diabetic macular edema. Diabetic macular edema is considered as a component of diabetic retinopathy. Treatment of diabetic retinopathy invariably affects diabetic macular edema and vice versa.

Another preferred utility of the present invention is a method for treating the diabetic macular edema component of diabetic retinopathy in a human patient.

Another preferred utility is the use in the preparation of a medicament for treating the diabetic macular edema component of diabetic retinopathy in a human patient.

Another preferred utility of the present invention is in treatment of uveitis.

Another preferred utility of the present invention is in treatment of macular degeneration. A further preferred utility of the present invention is in treatment of age-related macular degeneration.

The invention and its practice are further described, for illustrative purposes, in the following specific example.

### EXAMPLE

15 adult laboratory rats (Sprague-Dawley) were injected intraperitoneally on day 1 with SZT, 65 mg/kg, to render them hyperglycemic and to induce diabetes. 5 additional rats were treated with a similar volume of saline, to create a non-diabetic control group. Three hours prior to the STZ injection, and daily thereafter for a total of 6 days, 8 of the diabetic rats received an intramuscular injection, in the thigh muscle, of a suspension of unilamellar liposomes in saline comprising 75% 1-palmitoyl-2-oleoly-sn-glycero-3-phosphoglycerol (POPG) and 25% 1-palmitoyl-2-oleoyl-sn-glycero-3-phosphocholine (POPC), by weight, made by known extrusion methods, each injection consisting of a volume of 0.11 ml from a suspension containing 1.2×10⁷ liposome bodies per ml. The liposome injections to the animals of the diabetic+treatment group took place daily, on days 1 through 6. 7 of the diabetic animals received similar injections of the same volume of saline, according to the same dosage schedule. Animals of the non-diabetic control group received no injections, and remained normogylcemic.

On day 14, all animals from the three groups were sacrificed, and their eyes surgically removed and retinal tissue isolated from them. Tissue from one eye of each animal was processed for RNA isolation and analysed by qRT-PCR for cytokines IL-1, IL-6, TNF-α, all of whom are inflammatory cytokines, and MCP-1, an inflammatory chemokine. The RNA isolated was also analysed by qRT-PCR for anti-inflammatory cytokines TGF-β2 and IL-10.

In a repeat experiment, the same conditions were used except that the rats were injected with 0.15 ml of the suspension containing 1.2×10⁷ liposome bodies per ml.

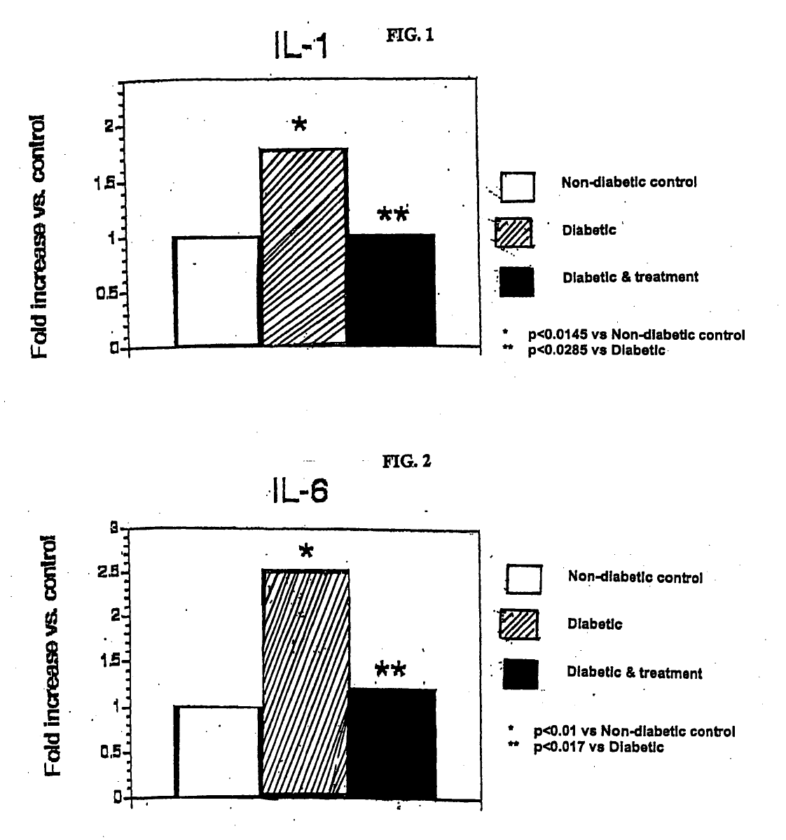

FIG. 1 of the accompanying drawings shows graphically the results of IL-1β mRNA measurements, averaged over all the samples of the respective groups. The vertical scale is the relative fold increase in cytokine mRNA in tissue, with the result from the untreated control group given a value of 1. It will be noted that the fold increase in IL-1β mRNA levels from the diabetic group is about 1.8 times that of the non-diabetic control animals (p<0.0145), whereas the value from the diabetic+treatment group of animals is the same as that from the non-diabetic control animals and is much less than that from the diabetic animals (p<0.0285). In a repeat experiment, the mRNA levels found in the diabetic+treatment group was significantly less (p=0.042) than that of the diabetic group.

FIG. 2 of the accompanying drawings similarly presents the results of mRNA measurements of the inflammatory cytokine IL-6. In this case, there is a 2.5 fold increase in the mRNA levels for this cytokine in the diabetic group compared with the non-diabetic control group (p<0.01) whereas the mRNA levels found in the diabetic+treatment group is reduced to about 1.2 times that of the non-diabetic control group and significantly less (p<0.017) than that of the diabetic group. In a repeat experiment, the mRNA levels found in the diabetic+treatment group showed a trend towards decrease in mRNA levels in comparison to that of the diabetic group although the decrease in mRNA levels did not reach statistical significance.

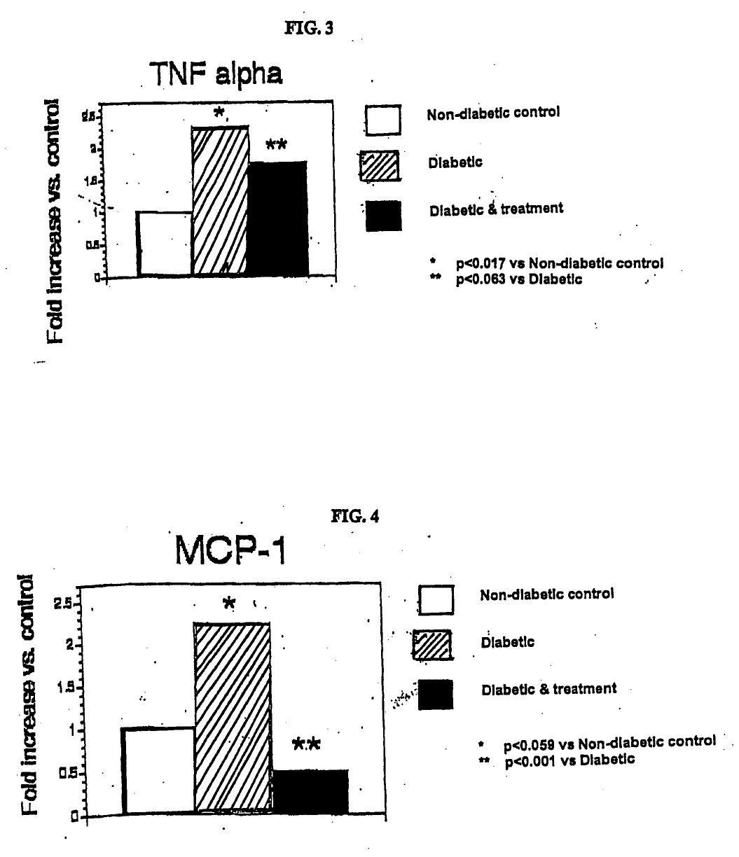

FIG. 3 of the accompanying drawings similarly presents the results of mRNA measurements of the inflammatory cytokine TNF-α. In this case, there is a 2.3 fold increase in the mRNA levels for this cytokine in the diabetic group compared with the non-diabetic control group (p<0.017) whereas the mRNA levels found in the diabetic+treatment group is reduced to about 1.7 times that of the non-diabetic control group and a trend towards lower mRNA levels than that of the diabetic group (p=0.063). In a repeat experiment, the mRNA levels found in the diabetic+treatment group showed a trend towards lower mRNA levels (p=0.054) than that of the diabetic group.

FIG. 4 of the accompanying drawings similarly presents the results of mRNA measurements of the inflammatory chemokine MCP-1. In this case, there is a 2.25 fold increase in the mRNA levels of this chemokine in the diabetic group compared with the non-diabetic control group (p=0.059) whereas the mRNA levels found in the diabetic+treatment group is reduced to about 0.2 times that of the non-diabetic control group and significantly less (p<0.001) than that of the diabetic group. In a repeat experiment, the mRNA levels found in the diabetic+treatment group was significantly less (p=0.0445) than that of the diabetic group.

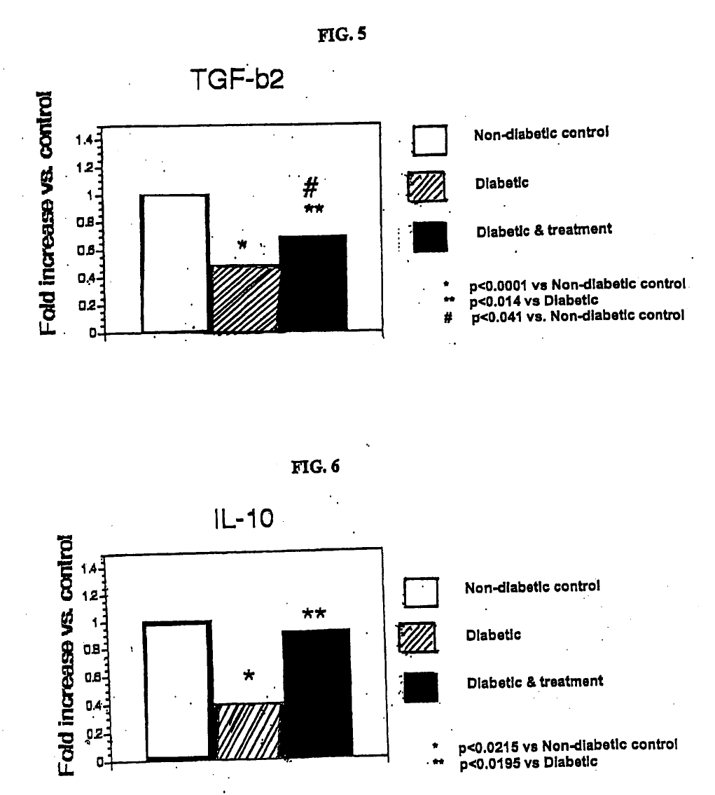

FIG. 5 of the accompanying drawings similarly presents the results of mRNA measurements of the anti-inflammatory cytokine TGF-β2. In this case, the diabetic group showed a significant decrease in the levels of TGF-β2 mRNA in comparison to the non-diabetic control group (p<0.001). There was also a significant decrease in the levels of TGF-β2 mRNA in the diabetic+treatment group in comparison with the non-diabetic control group (p<0.041) but there was a significant increase in the levels of TGF-β2 mRNA in the diabetic+treatment group when compared with the diabetic group (p<0.014). In a repeat experiment, the mRNA levels found in the diabetic+treatment group showed a trend towards increased levels in comparison to that of the diabetic group but the increase did not reach statistical significance.

FIG. 6 of the accompanying drawings similarly presents the results of mRNA measurements of the anti-inflammatory cytokine IL-10. In this case, there was about a significant decrease in the levels of IL-10 mRNA in the diabetic group in comparison with the non-diabetic control (p<0.0215). However, there was a significant increase in the levels of IL-10 mRNA in the diabetic+treatment group in comparison with the diabetic group (p<0.0195) while there was no significant difference in the relative expression of IL-10 between the non-diabetic control and diabetic+treatment groups. In a repeat experiment, the mRNA levels found in the diabetic+treatment group was significantly increased (p=0.0315) in comparison to that of the diabetic group.

The results shown in these experiments, conducted on a standard, accepted model of diabetic retinopathy, clearly indicate a down regulation of expression of mRNA of harmful inflammatory cytokines and chemokines, as well as an increase in the expression of mRNA of anti-inflammatory cytokines in retinal tissue of diabetic mammals, indicating utility in the treatment of diabetic retinopathy, uveitis, macular degeneration, and other inflammatory and/or vascular disorders of the eye in human patients in accordance with the present invention.

What is claimed is:

1. A process of retarding the development and/or progression of an inflammatory and/or vascular disorder in the eye in a mammalian patient, which comprises administering to the patient an effective amount of pharmaceutically acceptable phosphate-glycerol group presenting bodies, of a size resembling that of apoptotic cells or apoptotic bodies.

2. The process of claim 1 wherein the bodies are liposomes.

3. The process of claim 2 wherein the liposomes comprise from 60%-100% by weight of phosphatidylglycerol.

4. The process of any of claims 1 to 3 wherein the bodies have a diameter from about 20 nm to about 500 μm.

5. The process of any of claims 1 to 3 wherein the bodies are administered in a unit dosage amount of from about 500 to about 3×10¹⁴ bodies.

6. The process of any of claims 1 to 3 wherein the pharmaceutically acceptable bodies are administered systemically.

7. The process of any of claims 1 to 3 wherein the pharmaceutically acceptable bodies are administered intramuscularly.

8. The process of any of claims 1 to 3 wherein the pharmaceutically acceptable bodies are administered topically.

9. The process of any preceding claim wherein the disorder is diabetic retinopathy.

10. The process of any of claims 1 to 3 wherein the disorder is uveitis.

11. The process of any of claims 1 to 3 wherein the disorder is macular degeneration.

12. The process of claim 11 wherein the macular degeneration is age-related macular degeneration.

13. Use in the preparation or manufacture of a medicament for the treatment or prophylaxis of an inflammatory and/or vascular disorder in the eye in a mammalian patient, of pharmaceutically acceptable phosphate-glycerol group presenting bodies, of a size resembling the size of apoptotic cells or apoptotic bodies.

14. Use according to claim 13 wherein the bodies are liposomes.

15. Use according to claim 14 wherein the liposomes comprise from 60%-100% by weight of phosphatidylglycerol.

16. Use according to any of claims 13 to 15 wherein the bodies have a diameter from about 20 nm to about 500 μm.

17. Use according to any of claims 13 to 15 wherein the bodies are in a unit dosage amount of from about 500 to about 3×10¹⁴ bodies.

18. Use according to any of claims 13 to 15 wherein the pharmaceutically acceptable bodies are administered systemically.

19. Use according to any of claims 13 to 15 wherein the pharmaceutically acceptable bodies are administered intramuscularly.

20. Use according to any of claims 13 to 15 wherein the pharmaceutically acceptable bodies are administered topically.

21. Use according to any of claims 13 to 15 wherein the disorder is diabetic retinopathy.

22. Use according to any of claims 13 to 15 wherein the disorder is uveitis.

23. Use according to any of claims 13 to 15 wherein the disorder is macular degeneration.

24. Use according to claim 23 wherein the macular degeneration is age-related macular degeneration.

25. A method for treating the diabetic macular edema component of diabetic retinopathy in a human patient, which method comprises identifying a human patient exhibiting diabetic macular edema as a component of diabetic retinopathy, and administering to the patient an effective amount of pharmaceutically acceptable phosphate-glycerol group presenting bodies, of a size resembling that of apoptotic cells or apoptotic bodies.

26. The method according to claim 25 wherein the bodies are liposomes.

27. The method according to claim 26 wherein the liposomes comprise from 60%-100% by weight of phosphatidylglycerol.

28. The method according to any of claims 25 to 27 wherein the bodies have a diameter from about 20 nm to about 500 μm.

29. The method according to any of claims 25 to 27 wherein the bodies are in a unit dosage amount of from about 500 to about 3×10¹⁴ bodies.

30. The method according to any of claims 25 to 27 wherein the pharmaceutically acceptable bodies are administered systemically.

31. The method according to any of claims 25 to 27 wherein the pharmaceutically acceptable bodies are administered intramuscularly.

32. The method according to any of claims 25 to 27 wherein the pharmaceutically acceptable bodies are administered topically.
